(19) United States
(12) Patent Application Publication
(10) Pub. No.: US 2024/0307636 A1
(43) Pub. Date: Sep. 19, 2024

(54) Interactive Portable Vaporizer

(71) Applicants: Anthony Basso, Newport News, VA (US); Andrew Adcock, Appleton, WI (US); Monty Lee Savard, Beltsville, MD (US)

(72) Inventors: Anthony Basso, Newport News, VA (US); Andrew Adcock, Appleton, WI (US); Monty Lee Savard, Beltsville, MD (US)

(21) Appl. No.: 18/442,035

(22) Filed: Feb. 14, 2024

Related U.S. Application Data
(60) Provisional application No. 63/445,514, filed on Feb. 14, 2023.

(51) Int. Cl. A61M 11/04; G16H 40/63; G16H 70/00

(52) U.S. Cl. CPC A61M 11/042; G16H 40/63; G16H 70/00; A61M 2202/0007; A61M 2205/12; A61M 2205/18; A61M 2205/3561; A61M 2205/3576; A61M 2205/3584; A61M 2205/3653; A61M 2205/502; A61M 2205/8206; A61M 2205/8237; A61M 2209/086

(57) ABSTRACT

An interactive vaporizer system using a portable vaporizer in communication with an electronic device running a computer application enabling a user to manage vaporate usage and dosage. The system includes devices and methods for tracking quantity, composition, purpose, and effects of vaporates, managing duration of vaporizing sessions and frequency of draws, and monitoring results of vaporizing sessions to optimize future uses. The system includes a mouthpiece unit, cartridge unit, heating unit, and electronics unit as well as a docking unit for charging and weighing. The computer application provides access to an information database stored locally or remotely, the application captures vaporizing session information through user and vaporizer input that is stored to update the database, the application communicates information with the vaporizer, and the captured information can be shared with third parties.

### FIELD OF THE INVENTION

The invention discloses an interactive vaporizer system to monitor and provide feedback on usage.

### SUMMARY OF THE INVENTION

An interactive vaporizer system using a portable vaporizer in communication with an electronic device running a computer application enabling a user to manage vaporate usage and dosage. The system includes devices and methods for tracking quantity, composition, purpose, and effects of vaporates, managing duration of vaporizing sessions and frequency of draws, and monitoring results of vaporizing sessions to optimize future uses. The system includes a vaporizer having a microprocessor and wireless communication capabilities and an electronic device running a program to transmit user defined parameters to the microprocessor of the vaporizer and receive communications from the vaporizer. The vaporizer has a mouthpiece unit; a removable vaporate cartridge unit containing at least one chamber configured to hold vaporate; a heating unit containing at least one oven, each of the at least one oven being controlled by the microprocessor and/or computer application; an electronics unit having a rechargeable battery, a printed circuit board, and the microprocessor controlling the heating unit and communicating with and send and receive the electronic device; and a docking unit having a base that includes a weighing station and a recharging station, the docking station having the capability to wirelessly communicate with electronic device. The computer application provides access to an information database stored locally or remotely and the application is user programmed with vaporate specifics and the specifics are communicated with the vaporizer and stored to update the database.

### BRIEF DESCRIPTION OF THE DRAWINGS

These and other objects, features, advantages and aspects of the present invention can be better understood with reference to the following detailed description of the preferred embodiments when read in conjunction with the appended drawing figures.

FIG. 1 is a plan view of the vaporizing system showing the vaporizer inserted into the scale/recharger and cell phone, in accordance with the invention;

FIG. 2 is a plan view of the assembled vaporizer, in accordance with the invention;

FIG. 3 is an exploded view of the elements of the vaporizer in accordance with the invention;

FIG. 4 is an exploded view of the mouthpiece unit of the vaporizer of FIG. 2, in accordance with the invention;

FIG. 5 is an exploded view of the vaporate chamber, in accordance with the invention;

FIG. 6 is a exploded view the elements of the vaporizer, in accordance with the invention;

FIG. 7 is a perspective exploded view of the mouthpiece unit, in accordance with the invention;

FIG. 8 is a perspective view of the mouthpiece unit, in accordance with the invention;

FIG. 9 is an perspective view of the assembled mouthpiece unit, in accordance with the invention;

FIG. 10 is a view of the vaporate cartridge, in accordance with the invention;

FIG. 11 is an exploded view of the vaporate cartridge and screen, in accordance with the invention;

FIG. 12 is a perspective view of the alignment shaft, in accordance with the invention;

FIG. 13 is an exploded view of the heating unit and assembly flow, in accordance with the invention;

FIG. 14a is an exploded view of the distal elements of the heating unit, in accordance with the invention;

FIG. 14b is an exploded, partially assembled view of the distal elements of the heating unit, in accordance with the invention;

FIG. 14c is a perspective view of the assembled distal elements of the heating unit, in accordance with the invention;

FIG. 15 is an exploded view of the mounting of the heating coils, in accordance with the invention;

FIG. 16 is a view of the heating unit showing the heating coils held within the oven posts, in accordance with the invention;

FIG. 17 is an exploded view of the proximal elements of the heating unit, in accordance with the invention;

FIG. 18 is a perspective view of the assembled heating unit, in accordance with the invention;

FIG. 19 is a top view of the assembled heating unit showing the ovens, in accordance with the invention;

FIG. 20 is an exploded view of the electronics unit, in accordance with the invention;

FIG. 21 is a view of the weighted based, in accordance with the invention;

FIG. 22 is a view of the assembled water filter accessory, in accordance with the invention;

FIG. 23 is an exploded view of FIG. 22, in accordance with the invention;

FIG. 24 is an exploded view of the parts of the water filter accessory, in accordance with the invention;

FIG. 25 is a plan view of the docking station, with the vaporizer and vaporate cartridge in place, in accordance with the invention;

FIG. 26 is an exploded view of the vaporizer and vaporate cartridge removed from the docking station, in accordance with the invention;

FIG. 27 is an exploded view of the interior elements in the docking station; in accordance with the invention;

FIG. 28 is a view of the water filter accessory in an interactive base, in accordance with the invention;

FIG. 29 is an view of the water filter accessory in an interactive base, in accordance with the invention;

FIG. 29b is an exploded view of the interactive base, in accordance with the invention;

FIG. 30A is a flow chart of the system, in accordance with the invention;

FIG. 30B is a flow chart of the communications screen, in accordance with the invention;

FIG. 31A is a flow chart alter of an alternate screen, in accordance with the invention;

FIG. 32 is a screen indicating various strains, in accordance with the invention;

FIG. 33 is a screen with background on the vaporate, in accordance with the invention;

FIG. 34 is a user information screen, in accordance with the invention;

FIG. 35 is a user screen to create a session, in accordance with the invention;

FIG. 36 is a start session screen, in accordance with the invention;

FIG. 37 is a screen listing sessions, in accordance with the invention; and

FIG. 38 is an alternate home screen, in accordance with the invention.

### DETAILED DESCRIPTION OF THE INVENTION

### Definitions

As used herein the term “vaporate” shall refer to any substance that can be vaporized including but not limited to tobacco, cannabis, inhalable insulin, v-meds, eucalyptus oil, etc.

As used herein the term “electronic device” shall refer to any programmable device connected to the vaporizer, including but not limited to desk top computer, phone, tablet, etc.

As used herein, the term “vaporizing session” or “session” shall refer to the act of using the disclosed vaporizer to vaporize a singular vaporate to achieve a particular end desired by the user.

As used herein, the terms “draw” and “puff” are synonymously and shall refer to the act of using the mouth and breath to control the flow of vapor from the disclosed vaporizer.

As used herein, the term “mind and body agent” shall refer to a substance that has a mental and/or physical effect on a user upon ingestion. Ingestion methods including but not limited to vaporize, edibles, tinctures, infused liquids, topicals, sublinguals, suppositories, and vaginal applications. Mind and body agents can be medicinal or recreational substances.

As used herein, the term “PCB” shall refer to a printed circuit board.

### COMPONENT LIST

100 mouthpiece unit
110 mouthpiece
112 mouthpiece rim
114 inhalation outlet
120 flange magnet
122 diffuser bore
130 diffuser
132 diffuser flange
134 vents
136 V-shaped diffuser groove
140 mouthpiece gasket
142 mouthpiece gasket bore
144 mouthpiece gasket v-shaped groove
146 mouthpiece gasket vents
200 cartridge unit
210 vaporate cartridge
212 vaporate chambers
214 bar code
216 vaporate bore
218 vaporate groove
220 screen
222 screen bore
224 screen groove
300 heating unit
306 locking magnet
310 alignment shaft
312 alignment shaft body
314 V-shaped key
314a key distal end
315 shoulder
316 alignment shaft distal end
317 stationary ring
318 shaft cavity
320 retainer member
322 oven control PCB
324 oven control bore
326 oven control v-groove
330 spacing ring
340 connectors (oven PCB to oven post)
342 oven PCB transistors
350 insulating disk receiving holes
352 oven posts
353 oven post proximal ends
354 Oven nuts
356 insulating disk bore
358 disk groove
360 insulating disk
361 gasket
362 post screws
370 oven coils
371 oven coils proximal end
372 oven coils distal end
378 shaft retaining member
380 venturi disk
381 venturi central bore
382 venturi cavities
384 venturi threaded screw holes
385 top clamp
386 top clamp bore
387 top clamp screw holes
388 ovens
389 top clamp vent holes
390 Crash gasket
391 crash gasket bore
392 O-ring
393 gasket grooves
394 crash gasket vent holes
398 top clamp screws
400 Body unit
410 body
412 on/off button
416 body screw hole
500 electronics unit
510 electronics PCB
511 U-shaped base
512 battery
520 end cap
522 end cap screw hole
526 end cap screw
528 body screw
600 weighted base
610 weighted base retainer magnet
620 weighted base body
800 docking station
802 recharging station
840 weighing station
842 cartridge receiver
844 Deflection arm
846 Contact
848 raised flange
850 scale PCB plate
852 scale PCB
856 scale PCB receiving holes
860 scale bottom plate
862 scale plate receiving holes
866 scale plate retaining screws
900-999 computer application flowcharts
1000 vaporizing system
1010 vaporizer
1030 water filter accessory
1032 riser pipe mouthpiece
1034 bubbler pipe
1036 watertight base
1038 water filter magnet
1040 water filter body
1042 water filter gasket
1050 electronic device
1200 smart base
1202 control mechanism
1204 control support
1206 control screws
1220 smart base body
1230 smart base battery
1240 smart base PCB
1250 smart base plate
1260 smart base plate screws

### System

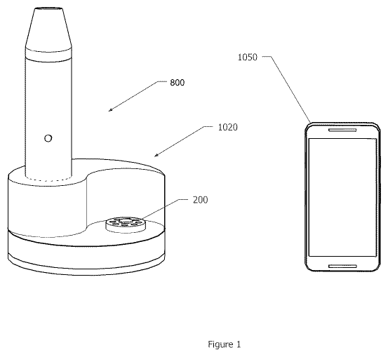

The disclosed vaporizing system 1000, an example of which is illustrated in FIG. 1, enables the user to control and track quantity, composition, purpose, and characteristic effects of a chosen vaporate, manage the frequency of draws and the duration of a vaporizing session, as well as monitor the results to help calculate the next dose. The calculations are based on a predetermined plan that can be developed, monitored, and updated by a user or, in some instances, by a physician. The vaporizing system 1000 consists of a vaporizer 1010, cell phone or other electronic device 1050, and a docking station 800 combination, all of which interact through a user-programmable computer application to communicate with one another.

Due to the number of parts involved with the construction of the disclosed vaporizer 1010, the elements have been broken down into five (5) sections, each of which has been broken down into individual components and described in greater detail.

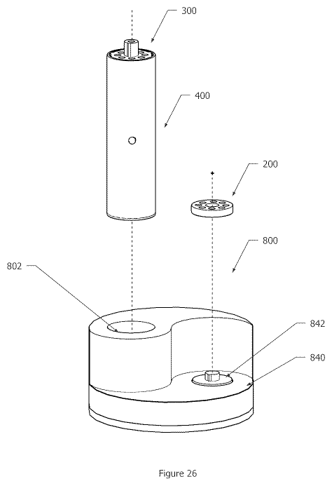

The example configuration of the docking station 800 includes a recharging station 802 and a weighing station 840 that allows for the positioning of the scale 820 within the weighing station 840 beneath the cartridge receiver 842 as will be disclosed in more detail in FIGS. 25-26b. The scale 820 is tared to the weight of the vaporate cartridge 210 and weighs the quantity of vaporate contained within the vaporate cartridge 210 both before and after use, automatically transferring the weights to the electronic device 1050. The docking station 800 will also contain a bar code reader and/or an RF chip reader that will automatically identify the vaporate cartridge's bar code and/or RF ID chip and automatically transfer that identification data to electronic device 1050. The electronic device then uses a computer application to save this ID and mass data for calculation of dosage, study usage data trends, and aid in selecting vaporates for specific user needs.

### Apparatus

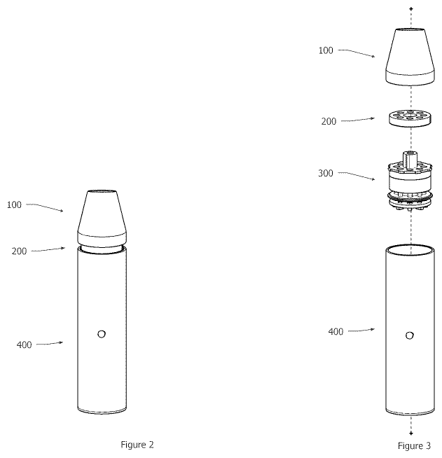

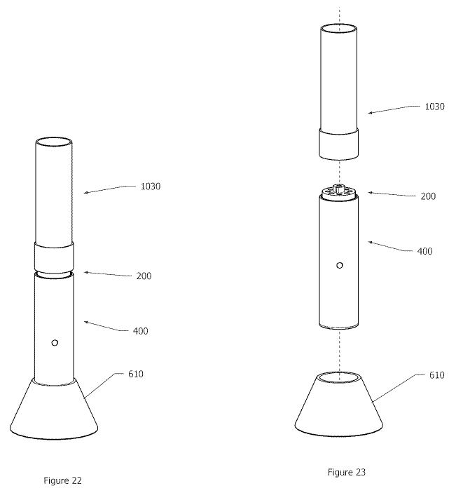

Vaporizer 1010 is comprised of five sections, namely the mouthpiece unit 100, the cartridge unit 200, the heating unit 300, the body unit 400, and the electronic unit 500. The assembled vaporizer 1010, an example of which is illustrated in FIG. 2, shows the mouthpiece unit 100, cartridge unit 200 and body unit 400 of the vaporizer 1010. The dimensions of the assembled vaporizer 1010 are dependent upon end user and application. The basic range would be an overall length of 4 to 8 inches with a cartridge height of 0.25 to 1.0 inches and a diameter of 1.0-2.0 inches. The exterior of the vaporizer 1010 can be manufactured from either plastic or metal, with the appropriate insulation provided. In situations where the user would find it difficult to hold the vaporizer 1010, a weighted stabilizing base 600 (FIG. 21) can be incorporated to allow the vaporizer to stand independently of the user. Alternatively, the base diameter can be increased to increase the base stability for standing on a table. The vaporizer 1010 can also be set in the docking station 800 to maintain the upright position.

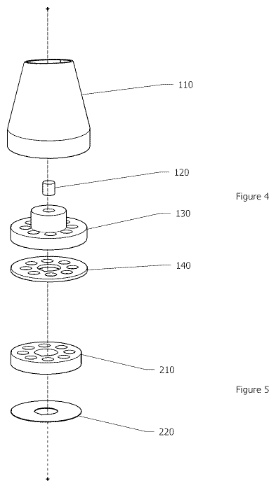

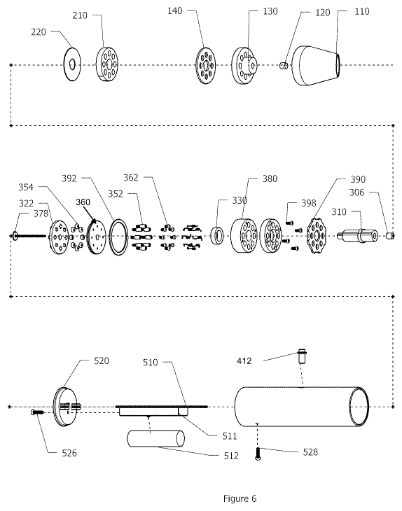

An example of the vaporizer unit 1010 is illustrated in exploded views in FIGS. 3-5. In FIG. 3 the vaporizer 1010 unit is illustrated with four (4) of the five (5) modular sections, namely mouthpiece unit 100, cartridge unit 200, heating unit 300, and body 400, more clearly illustrated. FIG. 4 shows components of the removable mouthpiece unit 100 in an assembly configuration including mouthpiece 110, magnet 120, diffuser 130, and mouthpiece gasket 140. The cartridge unit 200 illustrated in FIG. 5 shows the vaporate cartridge 210 and screen 220.

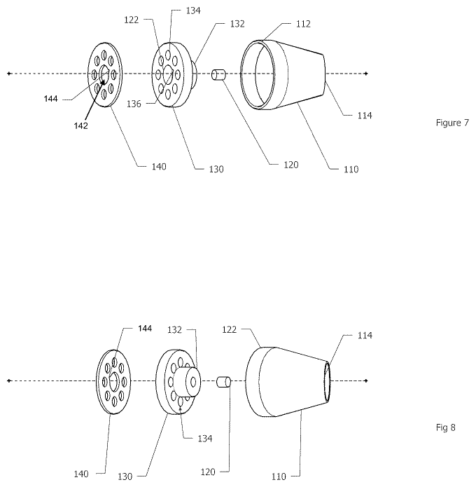

FIG. 6 shows an exploded view of all parts of the vaporizer 1010 to more easily understand the assembly. The numbering of FIG. 6 coordinates with the foregoing component listing as well as the detailed description of each vaporizer section hereinafter.

### Mouthpiece Unit

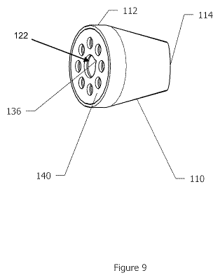

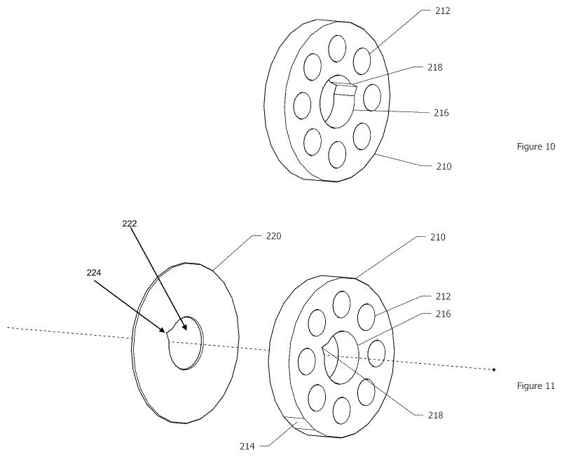

FIGS. 7 and 8 are exploded perspective view of the mouthpiece unit 100 with FIG. 9 illustrating the assembled mouthpiece unit 100. The removable mouthpiece 110 in the illustrated example is a circular frustum shape, providing a circular inhalation outlet 114. This configuration is an example and other configurations can be used as will be known to those skilled in the art. The material of manufacture for the mouthpiece 110 can be plastic, metal, or composite, with plastic or composite being preferred due to insulation qualities.

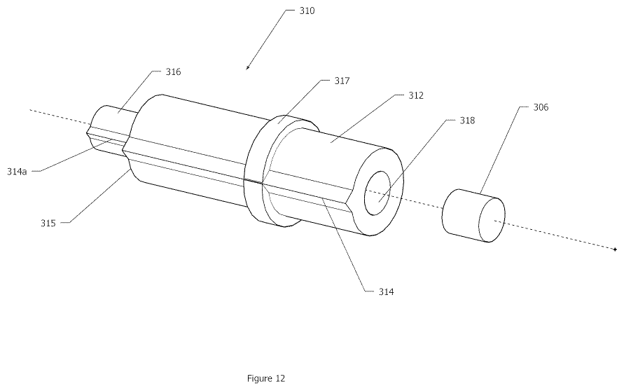

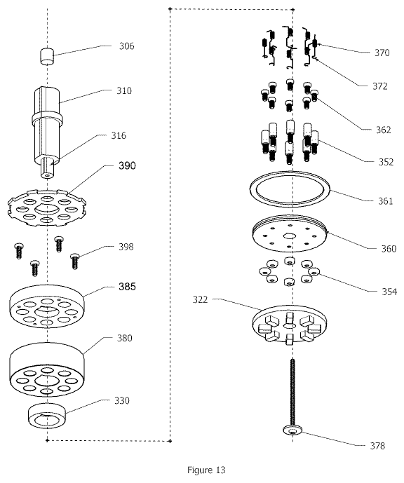

The mouthpiece rim 112 has a diameter and depth dimensioned to receive and enable the diffuser 130 to be recessed within in a press fit. The center of the diffuser 130 has a diffuser bore 122 that extends up to the flange 132 as described below. Cut into the diffuser bore 122 is a V-shaped diffuser groove 136 that is dimensioned to receive and align to the V-shaped key 314 of the alignment shaft 312 illustrated in FIG. 12. The diffuser 130 contains vents 134 that align with the vaporate chambers 212 (FIGS. 10-11) and serve to mix hot vaporate produced by one active oven with the cold air streams from all the inactive ovens, thereby dispersing the vaporate prior to entering the cone tip mouthpiece 110. Centered within the proximal side of the diffuser 130 is a bored diffuser flange 132 into which a flange magnet 120 is press fitted with the north pole pointing toward the inhalation outlet 114.

The mouthpiece gasket 140 contains the centered mouthpiece gasket bore 142 and mouthpiece gasket V-shaped groove 144 that enables alignment around the alignment shaft 312 shown in FIG. 12. The mouthpiece gasket 140 is also provided with mouthpiece gasket vents 146 to enable the vaporate to reach the inhalation outlet 114.

The ability to remove the mouthpiece 110 is important for hygienic purposes and therefore the mouthpiece is preferably manufactured from easily cleaned material. In the example embodiment illustrated herein the diffuser and the mouthpiece are connected magnetically, thereby making the elements easy to separate.

FIG. 9 illustrates the assembled mouthpiece 110 with the diffuser 130 recessed within the mouthpiece rim 112.

### Cartridge Unit

FIG. 10 illustrates the vaporate cartridge 210 within the cartridge unit 200, and FIG. 11 shows the vaporate cartridge 210 and screen 220 ready for assembly. The vaporate cartridge 210 contains vaporate chambers 212 extending through the proximal to distal sides and configured to receive the vaporate. The vaporate cartridge 210 will vary in thickness, diameter, and chamber size depending on the selected vaporate. The stainless steel, or equivalent, mesh gauge of the retainer screen 220 mesh also depends on the intended vaporate type. For example, a typical dry goods vaporate chamber 212 will hold a range of 0.1 grams to 0.5 grams of dried material and use a medium mesh screen 220 to retain that material. A wet goods vaporate chamber 212, however, will hold a range of 0.01 grams to 0.25 grams of oily or wax-like vaporate material and use a fine mesh screen 220 made for wicking.

Although eight (8) vaporate chambers 212 are illustrated for example purposes, the number, size, and distribution can vary dependent upon the vaporate as noted above. Each vaporate cartridge 210 may be identifiable by the scale, electronic device, and smart base by reading the bar code 214 and/or RF ID chip. As the vaporate cartridge 210 must be dishwasher or autoclave safe, barcodes 214 must be capable of undergoing dishwashing or autoclaving without damage. A vaporate cartridge 210 that does not need to be dishwasher or autoclave safe may use RF ID chip and/or barcode. The material of manufacture for the vaporate cartridges 210 is metal, ceramic, composite, or other equivalent material that meet the requirements set forth herein. Vaporate cartridges 210 are preferably fully reusable and recyclable if damaged. They are refillable by hand with a small funnel or dropper, or may come prefilled by retailers, or prefilled by conductors of studies.

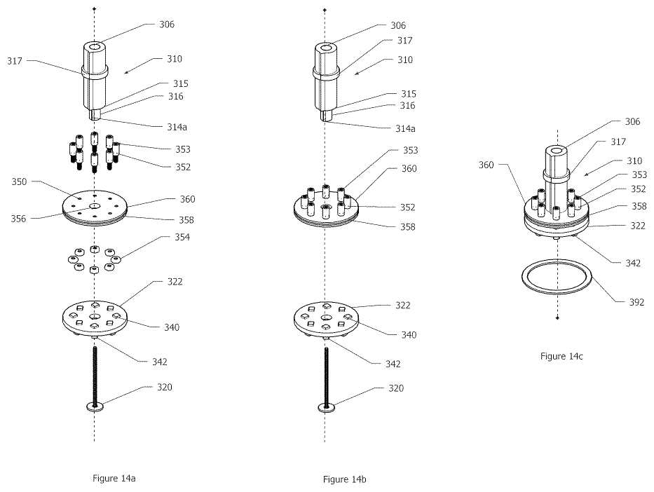

The screen 220, when used for dry goods vaporate, is manufactured from fine mesh stainless steel, or its equivalent, and is used to maintain the vaporate within the vaporate chambers 212, keeping the vaporate sequestered from the physical contact with the oven coils 370 (FIG. 13) below. The use of a fine mesh enables the air to flow through the vaporate chambers 212 to bring the smoke/vapor to the user. The screen 220 when used for wet goods vaporate, is manufactured from the same fine stainless steel mesh screen 220 with an individual pad of cotton above each oven for wicking. The mesh screen 220 and pad of cotton (not shown) are used to maintain the position of the vaporate within the vaporate chambers and sequestered from physical contact with the oven coils 370 below.

The vaporate cartridge 210 contains a vaporate bore 216 with a V-shaped vaporate groove 218 that is dimensioned to match the V-shaped diffuser groove 136 and diffuser bore 122. The dimensions of the V-shaped diffuser groove 136 and the vaporate bore 216 are matched in the screen bore 222 and V-shaped screen groove 224 to enable alignment with one another to receive the V-shaped key 314 and the alignment shaft body 312.

### Heating Unit

The alignment shaft 310 is illustrated in FIG. 12 and serves to maintain the various parts of the heating unit 300 aligned and adjacent to one another. The proximal end of the alignment shaft 310 contains a shaft cavity 318 dimensioned to receive a locking magnet 306. The locking magnet 306 is inserted, in this example, with the positive pole exposed to enable the flange magnet 120 and locking magnet 306 to maintain contact. If the pole of one magnet is switched the pole of the other magnet needs to be switched to prevent the magnets from repelling one another. A V-shaped key 314 runs along the length of the alignment shaft 310 and is dimensioned to be received within the screen groove 224, vaporate groove 218, and diffuser groove 136, as well as the grooves of the remaining elements described hereinafter. Likewise, the alignment shaft body 312 is dimensioned to be received within the screen bore 222, vaporate bore 216, and diffuser bore 122. The alignment shaft distal end 316 containing distal end V-shaped key 314a has a reduced diameter to provide more space on the oven control PCB 322 for ease of connection to the ends of the conductive oven posts 352. The reduced diameter also allows space for the head of shaft retaining member 378 to overlap with a circuit pathway on the oven control PCB 322 and uses the alignment shaft 314 as a conductor pathway back to the ovens 388. The distal end 316 is threaded to receive a retaining bolt, screw, or other removable retaining member 378.

The alignment shaft 310 additionally contains a stationary ring 317 to prevent element movement within the heating unit 300 and will be described in more detail hereinafter.

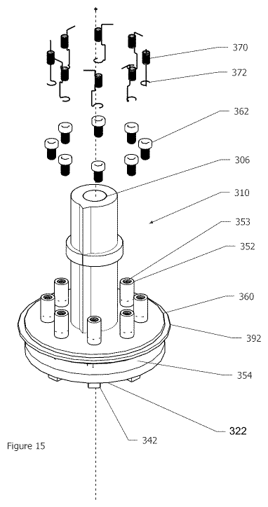

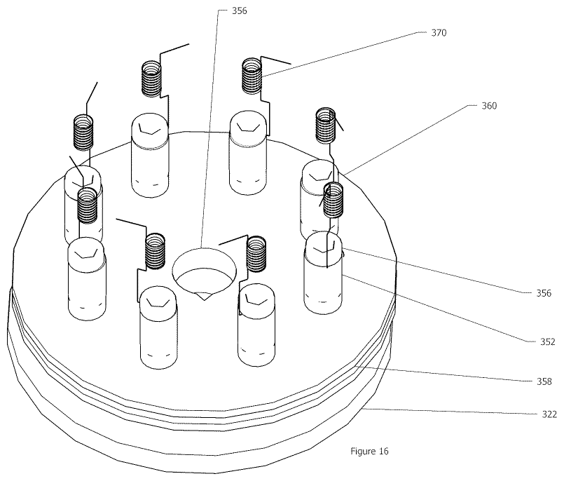

FIG. 13 is an exploded view of the oven unit 300, and FIGS. 14a, 14b and 14c illustrate the assembly series of this portion of the heating unit 300. The oven control PCB 322 has an oven control bore 324 with an oven control V-shaped groove (not illustrated) that maintains alignment with the remaining elements. In this portion of the heating unit 300 the individual oven PCB transistors 342 will align with the venturi cavities 382 containing the individual oven coils 370 as described hereinafter. This enables the heating of the vaporate to be controlled through the activation and deactivation of specific, individual ovens 388 (FIG. 19) by the oven PCB transistors 342 through use of the electronic device 1050. To prevent damage to any of the oven control PCB 322 elements, connectors 340 are placed on the proximal side of the oven control PCB board 322 to connect to the oven posts 352. The connectors 340 additionally prevent any unexpected overheating. A fusible link or similar overheat thermal sensor (not shown) will be located on the oven control PCB 322 for protection of the individual elements of the heating unit 300. The oven control PCB 322 will also contain a hall effect or similar magnetic flux sensor (not shown) that will prevent the oven coils 370 from being fired without the mouthpiece 110 or water filer accesory pipe attached.

In FIG. 14a the insulating disk 360 is positioned to receive the threaded oven posts 352 that extend through the insulating disk receiving holes 350. The oven posts 352 are secured in place by oven nuts 354. The use of the oven nuts 354 raises the insulating disk 360 away from the oven control PCB 322 to enable air flow.

The insulating disk bore 356 is dimensioned to receive the distal end 316 of the alignment shaft 310 with the insulating disk V-shaped groove 358 preventing rotation by interacting with the V-shaped key 314a. As illustrated in FIG. 14b the oven posts 352 have been secured to the insulating disk 360 through use of the corresponding oven nuts 354.

The insulating disk 360 slide along the reduced diameter of the alignment shaft 314a to abut the shoulder 315 shown in FIG. 14a created by the differential in diameter between the alignment shaft distal end 316 and the alignment shaft body 312. The shaft retaining member 378 is threaded into the threaded area (not shown) of the alignment shaft distal end 316 forcing the two parts together and into alignment. The wide head 320 of the shaft retaining member 378 forces the oven control PCB 322 and the insulating disk 360 together preventing separation or misalignment as illustrated in FIG. 14c. The gasket 361 will, when assembled, rest on the insulating disk 360 to provide additional insulation, sequester air flow and heat from the oven PCB transistors 342, and retain the oven unit 300 in the body 400. The insulating disk 360 is manufactured from thermoplastic, ceramic or equivalent insulating material and can be permanently adhered to the oven control PCB 322 by adhesives appropriated to the materials used.

With the shaft sized to a few thousandths of an inch smaller in length than the combined thickness of the disc and posts pressed together, the screw holds a small tension force, and the shaft holds a small compression counter force that pinches the discs together to maintain electrical contact between the electrical posts mounted to insulating disk 360 and the electrical conduction points on oven PCB 322.

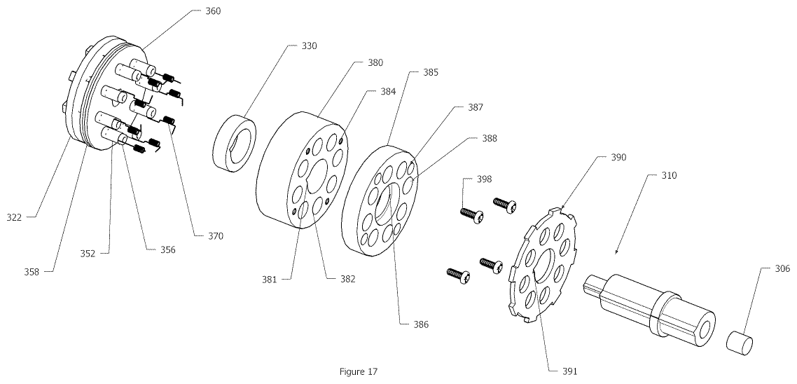

At time of manufacture the oven posts proximal ends 353 are threaded and dimensioned to receive post screws 362 as illustrated in the exploded view of FIG. 15. The oven coil distal ends 372 are looped to catch the ends of the post screws 362 and prevent the oven coils 370 from easily pulling out. The oven coils proximal ends 371 are bent at right angles to transfer the current as described hereinafter. FIG. 16 illustrates the oven coil 370 locked into the oven posts 352 by the post screws 362.

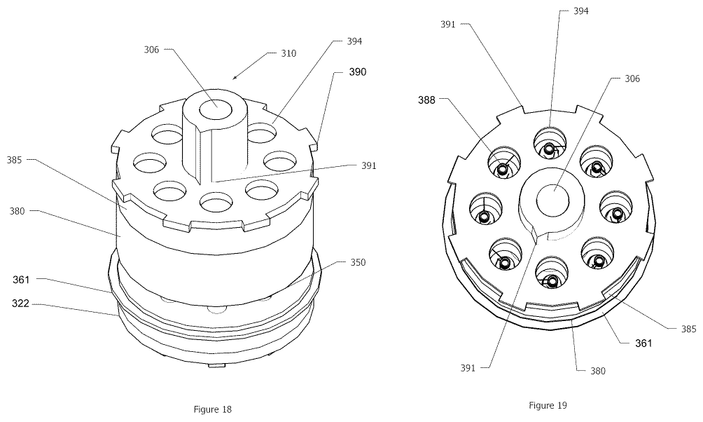

FIG. 17 illustrates the venturi disk 380, spacing ring 330, and top clamp 385 positioned to be placed over the oven coils 370 and oven posts 352. The spacing ring 330 is dimensioned to raise the venturi disk 380 above the oven posts 352 to enable the oven coils 370 to be positioned within the venturi cavities 382 to create the ovens 388. The top clamp screws 398 are received within the top clamp screw holes 387 and venturi screw holes 384 to secure the elements together. The crash gasket 390 and O-ring 392 are the placed over the top clamp 385 and secured thereto.

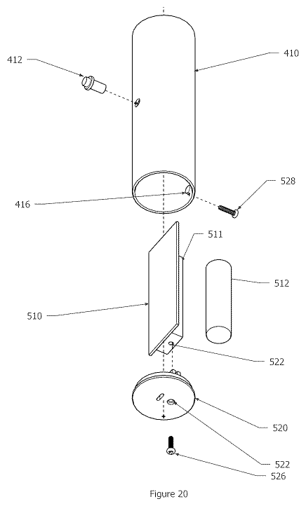

As seen in FIGS. 18 and 19 the venturi disk 380 has been placed over the oven posts 352 with the venturi cavities 382 receiving the oven posts 352 and oven coils 370. The venturi cavities 382 are dimensioned to receive the oven posts 352 without friction or excessive movement, and with sufficient clearance to allow air flow into the venturi cavities 382. The spacing between the oven posts 352 and the venturi cavities 382 prevents contact of both the oven posts 352 and oven coils 370. The oven coils proximal end 371 is bent to come in contact with the very top of the venturi disk 380 and is clamped to the venturi disk 380 with a top clamp 385 secured by top clamp screws 398. This clamping provides an electrical connection from each of the top of the oven coils 370, through the top clamp 385, into the alignment shaft 310, and back down to the oven control PCB 322, providing a complete electrical pathway and creating an oven 388 with one node for each oven 388 in parallel. The venturi disk 380 has a venturi central bore 381 as described with other elements that fit over the alignment shaft 310. There are four (4) venturi threaded screw holes 384 dimensioned to receive the top clamp screws 398. When assembled the top clamp 385 is placed on top of the venturi disk 380 and secured thereto with the top clamp screws 398. As with previously described elements the clamp center bore 386 forces the alignment of the top clamp vent holes 389 with the venturi vent holes 382.

The top clamp 385 is covered with a grooved crash gasket 390 with high temperature sealant, again the design of the crash gasket central bore 391 forces alignment of the crash gasket vent holes 394. An O-ring 392 is placed over the grooves 393 within the crash gasket 390 and fixed in place. The O-ring 392 needs to be retained adjacent the crash gasket 390 without the vaporate cartridge 210 being required to hold it in place when pressed down by the mouthpiece 100. The height provided by the O-ring 392 also spaces the oven unit 300 from the sides of the body 410 to aerate the air flow/insulation gap.

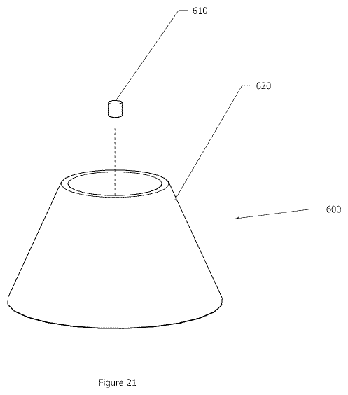

FIGS. 19 and 20 illustrate the assembled oven unit 300 with the oven coils 370 being seen within the venturi vent holes 382, top clamp vent holes 388 and crash gasket vent holes 394.

### Assembly

To assemble the heating unit 300, the elements proximal the vaporate cartridge 210 must be mounted prior to the distal to elements. The proximal elements are slid onto the alignment shaft 310, resting against the stationary ring 317 while the distal elements are slid onto the reduced diameter 316, abutting the alignment shaft shoulder 315.

It is beneficial however to have the distal element assembled and ready to slide onto the reduced diameter 316 of the alignment shaft 310. The oven posts 352 are placed through the insulating disk post receiving holes and secured with the oven nuts 354. The retaining member 320 can be placed through the oven control bore 324 of oven control PCB 322 with the remaining distal elements inserted onto the retaining element 320/378 or the final assembly can wait for the proximal elements. As the insulating disk bore and the oven control bore 324, they will be pressed up against the shoulder 315 of the alignment shaft 310.

Prior to mounting the elements onto the alignment shaft 310 the top clamp 385 can be secured to the venturi disk 380 using the top clamp screws 398. At the time of manufacture the venturi disk 380 has been threaded with screw holes 384 which, in conjunction with the top clamp screw holes 387 maintain the venturi cavities 382 and the top clamp vents 389 perfectly aligned. Alternatively, the alignment shaft 310 can be inserted, the top clamp 385 and the venturi disk 380 secured, thereby using the alignment shaft 310 to ensure alignment. Other methods can also be used to secure the two elements together and will be known to those skilled in the art.

The first element to be inserted onto the alignment shaft 310 is the crash gasket 390 which is maintained in the proximal position by stationary ring 317. After the crash gasket, the top clamp 385 and venturi disk 380 unit is inserted. Once these elements are on the alignment shaft 310 the spacer ring 330 is placed on the alignment shaft 310. The dimensioning of the spacer ring 330 must not only be such that its interior bore and V-shaped groove are dimensioned to be received by the alignment shaft but that the exterior diameter does not block the venturi cavities 382. Air flow is maintained with all elements by the alignment shaft 310. The height of the spacer ring 330 is critical as it serves two purposes. First to provide sufficient air flow space and second to place the oven coils 370 within the venturi cavities 382. Therefore, the spacer ring 330 has a height equal to the oven posts 352 and the heads of the post screws 362, placing the heater coils 370 within the venturi cavities 382.

With the proximal elements assembled onto the alignment shaft 310, the distal elements are assembled onto the lower part of the shaft as described above. Prior to placement of the distal elements onto the alignment shaft 310, an O-ring gasket 361 is placed on the insulating disk. The distal elements are slid onto the reduced diameter 316 of the alignment shaft 310 and the alignment shaft mounting screw 378 is threaded into the threaded receiving hole of the reduced diameter 316 of the alignment shaft 310. The extra wide head of the retaining member acts as a flange that forces the assembled parts together against the shoulder 315.

The length of the reduced diameter 316 of the alignment shaft 310 is a few thousandths of an inch less than the combined thickness of the oven control PCB 322, oven nuts, insulating disk 360, and oven posts 352 pressed together. The retaining member 320 holds a small tension force, and the reduced diameter 316 portion of the alignment shaft 310 holds a small compression counter force that pinches the disks together to maintain electrical contact between the oven posts 352 mounted to insulating disks 360 and the electrical conduction points on the oven transistors 342.

### Electronics Unit

The electronics unit 500, illustrated in FIG. 20, consists of the electronics PCB 510 that interacts with the electronic device 1050, controls the oven PCB 322 that controls the oven 370 coils, and interacts with the smart base accessory 1200 (FIG. 25). The interaction between the individual elements of the vaporizer system 1000 is disclosed hereinafter. The electronics PCB 510 is attached to a U-shaped base 511 to receive the rechargeable battery 512. The electronics PCB 510 and attached battery 512 are dimensioned to be received within the body 410 abutting the heating unit 300. U-shaped base 511 may have a base screw hole for receiving end cap screw 526. An end cap 520 contains an end cap screw hole 522 to receive the end screw 526. Once the PCB 510 has been inserted into the body 410, the end cap 520 is in place and the end cap screw 526 is tightened into the end cap screw hole 522, thereby enclosing the electronics unit 500 and heating unit 300 within the body 410. Also illustrated in this Figure is the on/off button 412.

As shown in FIG. 20, the heating unit is retained within the body 410 through securing screw 528 being threaded into threaded receiving hole 416. The ease of disassembly enables the vaporizer 1010 to be repaired and/or parts replaced. In this way the vaporizer, as well as other elements are environmentally sound by minimizing waste.

### Base Accessory

In FIGS. 21, 22, and 23 a weighted base 600 is provided to enable the user to place the vaporizer 400 in an upright position without contact. Most commonly the weighted base 600 is used in conjunction with the water filter accessory 1030. The mouthpiece unit 100 has been removed in order to receive the water filter accessory 1030

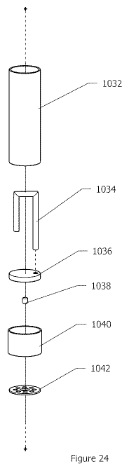

FIG. 22 shows water filter accessory 1030 latched to the vaporizer body 400, replacing the cone tip mouthpiece 100 and installed in the weighted base 600 used for stability. FIG. 23 shows an exploded view of water filter accessory 1030, vaporizer 400, and weighted base 600 to show the placement of the vaporate cartridge 210. Weighted base 600 comprises a body 620 that includes a retainer magnet 610 for added stability.

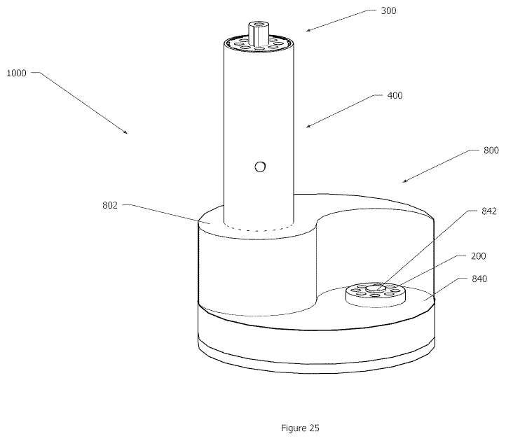

The riser pipe mouthpiece 1032 is illustrated in an exploded view in FIG. 24, wherein the bubbler pipe 1034 and watertight base 1036 seal together to form a water-tight zone that the vaporate must bubble through via the bubbler pipe 1034. The riser pipe mouthpiece 1032 is a tempered borosilicate glass tube or equivalent material and will bear an etched or painted line to show the correct water level to allow bubbling through the water but also to prevent accidently back siphoning of water into the vaporizer 1010.

Bubbler pipe 1034 is a hollow tube formed into a u-shape that is press fit and sealed into the watertight base 1036 to transmits the vaporate from below. The bubbler pipe 1034 extends up through the watertight base from the watertight base 1036 and continues to above the waterline. The bubbler pipe 1034 turns 180 degrees with a radius allowing it to fit within the riser pipe mouthpiece 1032 and continues back down below the water line, terminating with an open end.

The watertight base 1036 contains a bore to accept the press fit bubbler pipe 1034 and is sized to fit inside and seal against the riser pipe 1032. The distal, or dry side of watertight base 1036, contains a cavity to accept pressed fit magnet 1038 to provide magnetic latching to vaporizer 1010. A water filter body 1040 is sized to receive the riser pipe mouthpiece 1032 at the proximal end and sized to receive water filter gasket 1042 at the distal end and is attached to both elements with a sealant. The water filter body 1040 provides a negative space where the hot and cold oven air streams mix and diffuse.

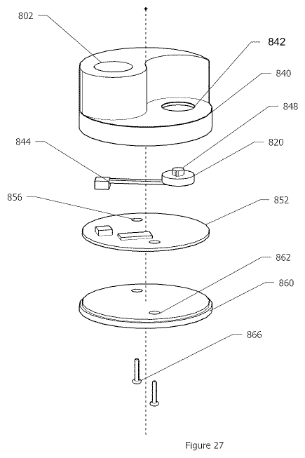

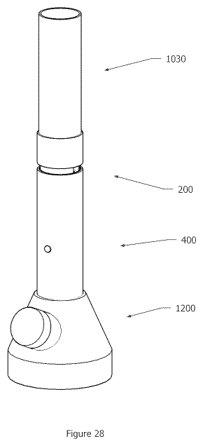

FIG. 27 shows a smart base accessory 1200 in assembled form for use with the vaporizer body 400 and water filter accessory 1030. The smart base 1200 connects to and interacts with the vaporizer main electronics PCB 510 to provide an additional user interface with a control mechanism 1202 that may be a combined screen/dial/button. FIG. 28 shows an exploded view of the smart base accessory 1200 in use with vaporizer body 400 and water filter accessory 1030 and vaporate cartridge 200.

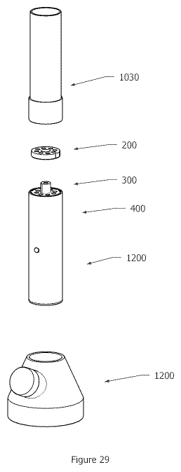

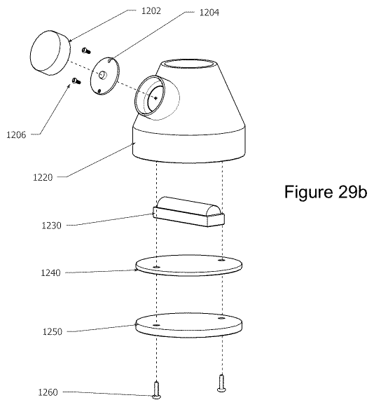

FIG. 29 shows an exploded diagram of smart base accessory 1200. The control mechanism 1202 can be used for menu browsing and selection, display of dosage data, adjusting of device settings, or displaying a static or moving images to display notifications or for general entertainment. A dial knob is shown in FIG. 29 by way of example, but any appropriate mechanism could be used including but not limited to buttons and touch screen. Screws 1206 mount the control mechanism 1202 to the support 1204 to the body 1220. Rechargeable battery 1230 is held in a u-shaped retainer and attached to Smart Base PCB 1240 to provide stand-alone power for the unit. Smart base PCB 1240 performs power management and recharges the battery 1230 with a USB charging port accessible by a small hole in the Smart Base body 1220. Smart base PCB 1240 connects to the vaporizer electronics PCB 510 through the USB port, and/or with wireless signal. Smart Base PCB 1240 is mounted to base plate 1250 and body 1220 with two screws 1260 that continue through Base Plate 1250, Smart Base PCB 1240, and into Body 1220.

### Docking Station

FIG. 25 illustrates the vaporizer body 400 placed in the recharging station 802 within docking station 800 and the cartridge unit 200 placed within the weighing station 840. As can be seen in this figure, the mouthpiece unit 100 has been removed, exposing the heating unit 300. In FIG. 26 the body 400 unit has been removed to clearly illustrate the entry to the recharging station 802. Additionally, the cartridge unit 200 has been removed to show the weighing station 840.

In FIG. 27 the example docking station 800 is shown in exploded view to illustrate the components. The cartridge receiver 842 is dimensioned to be received within the weighing station 840 and which houses scale 820 carried at the end of a deflection arm 844 having a contact 846. The cartridge receiver 842 has a raised flange 848 dimensioned to receive the vaporate bore 216 and screen bore 222, thereby preventing lateral movement during weighing. The scale PCB plate 850 connects to the scale 820 via the contact 846. The scale bottom plate 860 and scale PCB plate 850 both contain scale receiving holes 862 and 856 respectively dimensioned to receive the scale retaining screws 866. The screws 866 are received in reciprocating threaded holes within the weighing station 840 (not shown) to secure the unit.

The weighing station 840 is designed to provide for milligram accuracy when weighing dosages. The mass values are measured and reported to the electronic device for analysis and calculations, enabling monitoring of the vaporate quantity and type. As with any device generating heat, all elements should be insulated both within and along the exterior.

### Computer Application and Electronic Device

The vaporizer 1010 and the docking station 800 of the disclosed vaporizing system 1000 communicate with electronic device 1050 programmed with an application enabling a user to communicate with the vaporizer and to manage vaporate use. Electronics unit 500 within body 400 unit of vaporizer 1010 and docking station 800 each communicate with the application of electronic device 1050. Any communication means known in the art can be used by electronics unit 500 and docking station 800 including but not limited to Bluetooth wireless connectivity. Using the application, a user can control and track various vaporate features such as the quantity, composition, purpose, and characteristic effects of a chosen vaporate, as well as manage the frequency of draws and the duration of a vaporizing session, and monitor the results to help calculate and optimize future doses of vaporate. As the use of vaporates such as cannabis is becoming more widespread and accepted both recreationally and medically to alleviate physical and mental ailments, the ability to manage use, dosing, and effects is important. The application allows the user to explore vaporates with the vaporizer in a knowledgeable and responsible way. Further, the application may serve as a guide to mind and body agents enabling users to knowledgably and responsibly explore diverse ingestion methods such as edibles, tinctures, infused liquids, topicals, sublingual, suppositories, and vaginal applications in a personalized and informed manner.

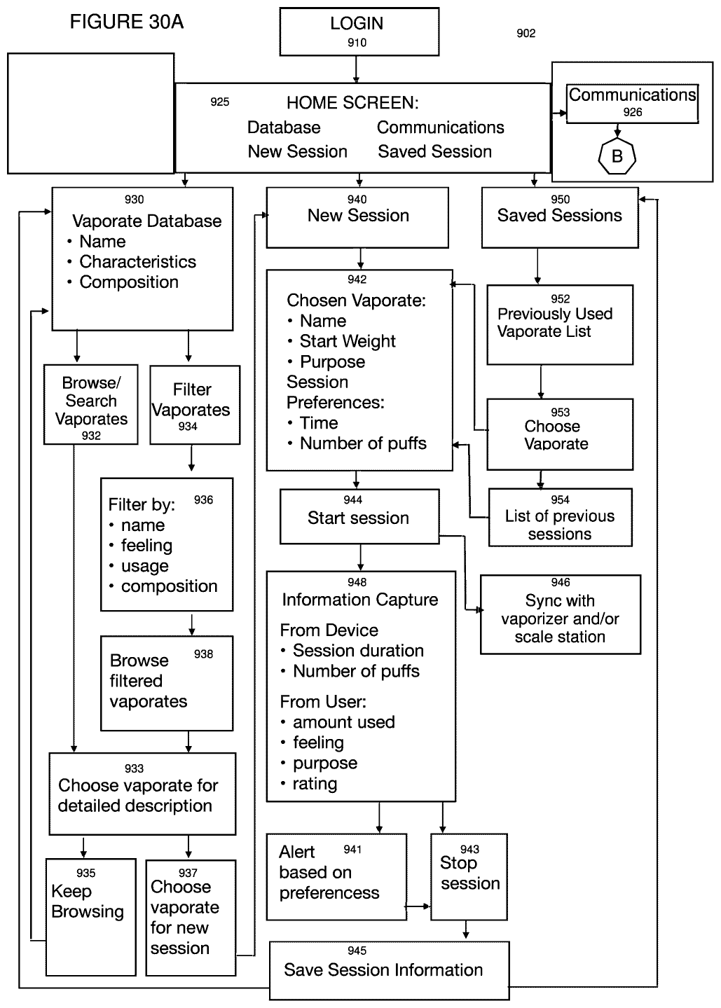

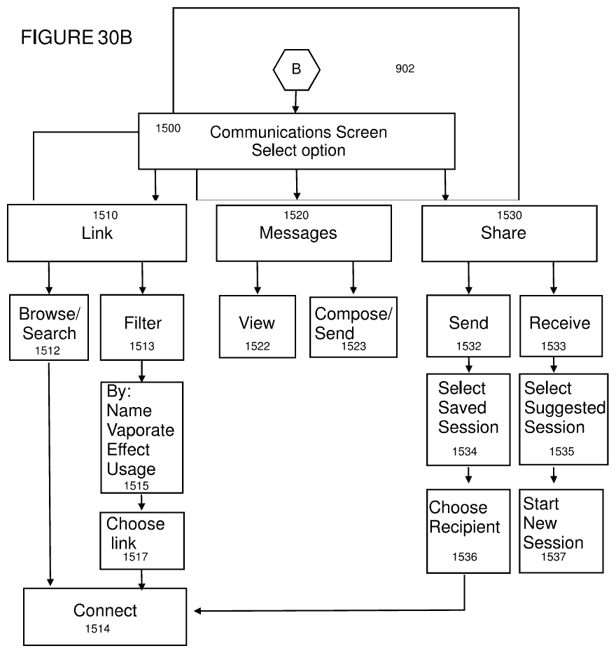

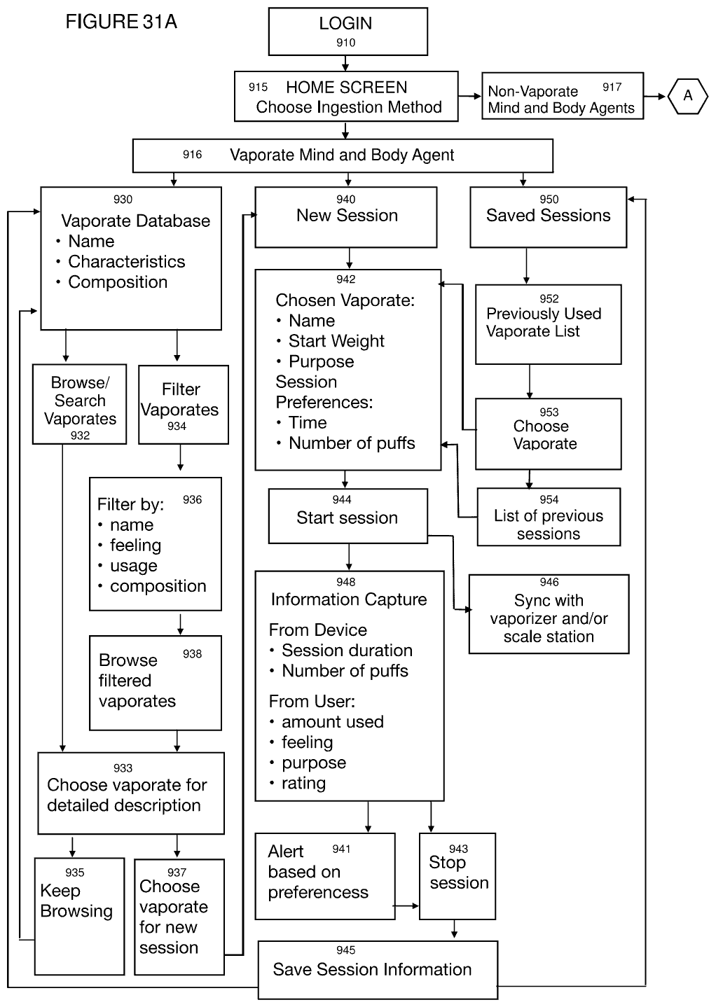

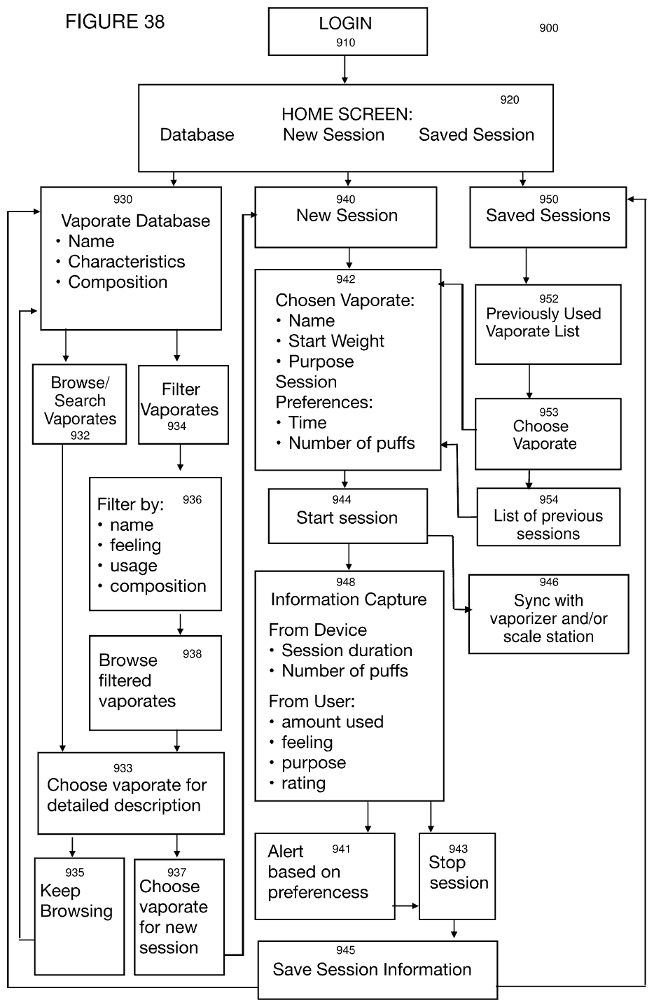

A flowchart 900 illustrating a computer application carrying out basic tasks of the vaporizer system 1000 is shown in FIG. 38. When the application is opened on an electronic device 1050, a user enters the application via a secure login screen 910. Upon successful login, the user is taken to a home screen 920 displaying options to search the vaporate database, create a new session, or revisit a saved session. These options allow a user to explore information about unknown vaporates, log new experiences with unfamiliar vaporates, and revisit previously used vaporates. As shown in FIGS. 30a and 30b and explained in more detail below, a further communications option may be available to share information with a health care provider or other interested parties. Such communications would be beneficial to receive care instructions and dosage suggestions as well as to share results, recommendations, experiences, and side effects. Further, as shown in FIGS. 31a and 31b, an ingestion methods option may be available to display various ingestion methods for mind and body agents including vaporize, edibles, tinctures, infused liquids, topicals, sublinguals, suppository, and vaginal applications. For all methods other than vaporize, the application will prompt the user for product information, and the user will be given access to the variety database along with subsequent options identical to those available for vaporate usage. Other useful options known to those skilled in the art can also be offered on the home screen.

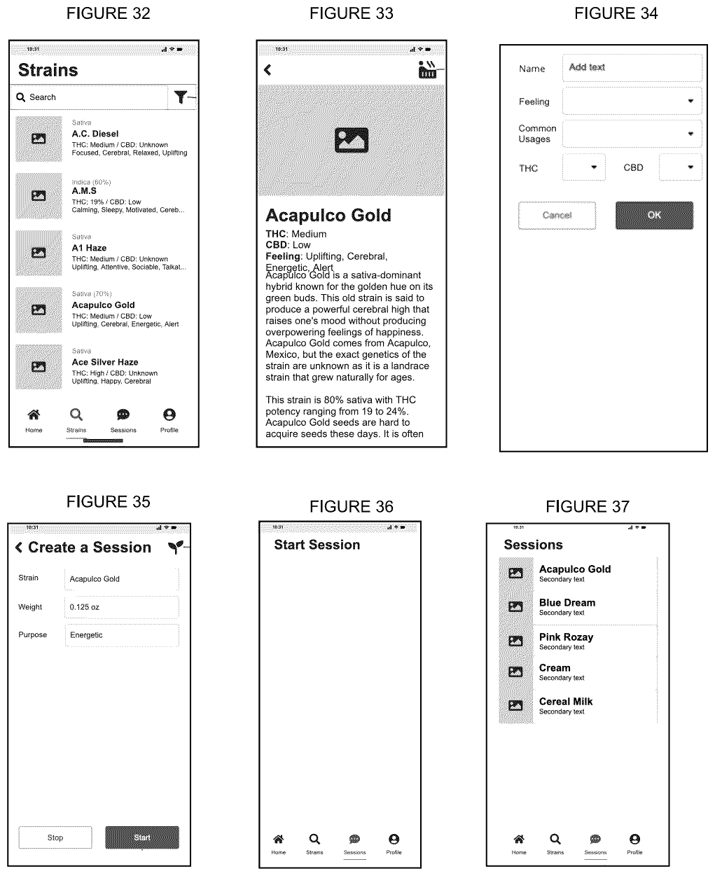

If a user selects the database 930, a vaporate list appears that can be browsed and searched 932 or filtered 934. The vaporate list contains a brief summary of each vaporate, for example the name, characteristics, and composition. As shown in FIG. 32, a cannabis strain list displays the product name, the strain type, the THC and CBD composition, and the common effects of the strain on users. A user can select a particular vaporate to access a more detailed description of the vaporate 933. As shown in FIG. 33 for example, a user has chosen Acapulco Gold from the vaporate list and the application now displays more information about the strain such as physical characteristics, source information, potency, common usages, and details about effects on the mind and body. The database can be stored remotely or locally on the application. The details provided for each vaporate will initially be populated on the application by typical effects, common uses, and known facts about the vaporate. As the user explores each vaporate and logs their own experiences, the display can update to include details personalized to the specific user. If a user chooses to use the vaporate displayed, the user can select the “create a session” option from the display page 935. If a user decides to not use the vaporate displayed, the user can go back to the vaporate list to resume browsing 937.

The vaporate list can be filtered using the filter option 934. When a user selects the filter option, the application displays a list of filter options 936. As shown in FIG. 34 for example, a cannabis strain database can be filtered by name, feeling provided to user, common usages of the strain, and THC/CBD composition. As noted above, the feeling provided to user and common usages would initially be typical effects and uses but would become personalized after updates by the user. Once the vaporate list is filtered based on the user's choices, a narrowed list is displayed by the application for the user to browse 938. As described above, the user can select a particular vaporate for more information 933 and then determine whether to use the vaporate and create a new session 935 or go back to the vaporate list to resume browsing 937.

A new session can be initiated from the vaporate database as noted above, or a new session can be selected from the home screen. With either option, when a new session is selected 940, the application opens a display to create a new session 942. With each new session created, the date and time are captured. As shown in FIG. 35 for example, the new session display for cannabis includes the strain name, the starting weight of the product being used, and the purpose of the strain. Further, the new session display may include other features such as but not limited to a duration preference, a draw preference, or a dosage preference. A duration preference would include any time limitation for the session desired by the user—for instance, if the user only wants the session to last 30 minutes. A draw preference would include a maximum number of draws the user wants to have during a session—for instance, if the user knows that 5 draws of a particular vaporate has the optimal effect on them. A dosage preference would include a draw per amount of time limitation—for instance, if a doctor prescribes 1 draw/hour to alleviate pain. All information inputted into the new session can be populated by initial database information, entered by a user, communicated by the vaporizer and scale, or a mix of all. A new session can be created for vaporates included in the database or for vaporates that are known only by the user. Also, the user may change the official name of a vaporate included in the database to a more common name or nickname known by the user. Further, in applications having a communications option, a healthcare provider could input information to create a session.

Once a new session is created 942, the session commences when the user selects the start option 944. Prior to starting a session, the user should prep the vaporizer by inserting the weighed vaporate and powering on the vaporizer. If an integrated scale is being used, the weight of the vaporate for a particular session is captured by the scale and communicated to the application. The vaporizer can be manually powered on or the application may communicate with the vaporizer to power on when the session is started. When the vaporizer is powered on and the session is started, the application syncs 946 with the vaporizer and scale if applicable during which any limitations set by the session are communicated to the vaporizer and any information gathered by the scale station is communicated to the application. Further, a start session display is provided by the application 948 to capture information communicated from the vaporizer 1010 or scale station 1020, an example of which is shown in FIG. 36. In the start session display, the user can record mental and physical effects experienced during the session. The start session display may also include a timer and notifications received from the vaporizer based on the set user preferences. For example, the application can display when a draw preference has been met or can provide an alert 941 when the next draw should be taken to fulfill a dosage recommendation. A new session is concluded 943 when at the user selects the stop option on the display. Alternately, the session can be terminated by the application based on session preferences and notifications received from the vaporizer. When a session is stopped, all information captured during that session is saved 945. The end weight of the vaporate is determined and the amount of vaporate used in a session is calculated and saved to the session information. The information recorded by the user during the session is saved to the vaporate database thus updating the database with personalized vaporate details. Further the session is saved to the application so that the session can be reviewed, revisited, recreated, and updated in the future.

Saved sessions enable a user to organize and optimize vaporate experiences. Sessions for particular ailments, moods, etc. can be easily located and recreated allowing the user to knowledgeably and responsibly care for themselves. Once a session is saved, all information recorded in that session becomes part of their vaporate database. Accordingly, a user can easily choose an appropriate vaporate to suit their condition by filtering the database. For example, users can filter by a particular effect they want to achieve, by an ailment they want to treat, or by a composition that they know works best for them. The more sessions that are recorded, the more personalized the application becomes for the user.

When saved sessions are selected from the home screen 950, the application displays a list of vaporates previously used by the user 952. As shown by example in FIG. 37, a display screen for saved cannabis session includes vaporte name and optional secondary text that can be input by the user. As with the database list described above, the user can select a particular vaporate 953 for more information about the vaporate and then determine whether to use the vaporate and start a session or go back to the saved vaporate list to resume browsing. Further, selecting a vaporate from the previously used vaporate list prompts the application to display a listing of sessions during which that vaporate was used 954. Starting a session from a saved vaporate follows the same process previously described for starting a new session 940. A user can choose to revisit a saved session or change aspects for a different experience. When revisiting a saved vaporate, a new session is created capturing the time and date as well as updates and any new preferences which will be saved to the session and the database. Accordingly, it is possible to have multiple sessions saved for one particular vaporate that differ depending on date, time, and the session preferences. These separate sessions for like vaporates can be compared and rated by the user.

When the application includes a communications option, the communications option can be selected from the home screen 925 as illustrated if FIGS. 30a and 30b. The communications option enables the user to communicate with healthcare providers and other interested parties. Using the communications option, the user can send and receive messages 1520 as well as share save sessions 1532 and received session recommendations 1533. For instance, if working with a healthcare provider to treat pain, the user can send a saved session to the provider for review. If the provider has any recommendations for changing vaporate, dosage, etc., the provider can create a recommended session. Upon receipt, the recommended session can be accepted by the user thus creating a new session that follows the previously described process. Further, outside of healthcare, sessions can be shared with third parties who may be interested in your experiences with particular vaporates. The communications option can also include a linking function for connecting with other application users 1510. Links can be made through browsing or filtering based on identifiers such as name, vaporate used, desired effects, and reason for usage. Once links are established, sessions can be shared between parties.

### Operation

Prior to a vaporizing session, a vaporate cartridge is weighed in the weighing station or the barcode of the cartridge is read to determine the starting weight. Once the cartridge is inserted into the vaporizer, the the vaporizer is powered on, and the electronics unit 500 processes any input communicated from the computer application or the docking station. Once the input is processed, the electronics unit will communicate with the oven control PCB 322 to prompt the heater unit to heat to the appropriate temperature for the selected vaporate. As the vaporate heats within the cartridge unit, the resulting vapor will travel through the vents within the vaporizer up through the mouthpiece unit. When a vaporizing session is complete and the vaporizer is returned to the docking station, the vaporate cartridge may be weighed to capture the end weight and relay that information to the computer application.

As the user draws on the mouthpiece, the vaporizer may monitor each draw and collect information in the microprocessor of the electronics unit. Relevant information gathered by the vaporizer such as number of draws and duration of vaporizing session may be communicated via wireless communication means such as Bluetooth by the electronics unit to the computer application.

### Synopsis

The pending vaporizing system features a synchronized interaction between the disclosed specialized vaporizer and an associated application. Setting up a user profile is an important first step for the application. Information such as height, weight, and any preexisting conditions will allow the application to determine dosage suggestions for the user.

Upon initiating the vaporizer, users will load a cartridge that is filled with a chosen vaporate. The user inputs specific vaporate details into the app, encompassing the known details. For this example cannabis will be used and the specific details would be the terpene type and percentage, phytocannabinoid specifics such as THC/CBD/CBDA percentages, and the strain name, thus creating a comprehensive strain profile. It should be noted that although cannabis is being used as an example here, the use is not limited to cannabis and includes, as noted heretofore, a number of other vaporates.

The application, integral to the system, guides users through a structured and informed session. Users are prompted to specify the session's purpose, differentiating between medicinal and recreational needs like chronic pain or sleeplessness, and anxiety and stress, with the app suggesting an optimal distribution amount in milligrams based on the user's profile information. Throughout the session, the app records essential details such as timestamp, date, distributed amount, and intervals between puffs, creating a detailed record of consumption habits.

Additionally, the app actively engages users with periodic prompts during the session, seeking qualitative insights that aid in determining the efficacy of the session, as well as helping the user identify the effects they are experiencing such as creativity, energy, sleepiness, or hunger. Post-session, the vaporizer's placement on the recharger involves a weight-sensing process, accurately measuring the remaining content in the cartridge. This system offers a comprehensive overview of the consumed terpene and phytocannabinoid content.

Notably, the application's post-session analysis provides users with summaries and recommendations, facilitating informed decisions for future sessions. The app will facilitate the user's qualitative experience and assist in identifying possible negative effects such as dry mouth, paranoia, or forgetfulness. This analytical feedback loop empowers users to optimize dosage control based on the nuanced interplay of terpenes and phytocannabinoids, resulting in a truly personalized and efficient cannabis experience that evolves with the user's preferences and needs over time.

The versatile disclosed system extends its functionality beyond vaporization to accommodate various cannabis ingestion methods, including edibles, tinctures, infused liquids, topicals, sublinguals, suppositories, and vaginal applications. Users navigate the application seamlessly to select their preferred ingestion method, initiating a user-friendly interface that mirrors the vaporization process.

For instance, when opting for edibles, users input details like the edible type, phytocannabinoid percentages and type, and desired effects into the app, establishing a personalized profile. The application then guides users through the consumption session, prompting them to identify the purpose, suggested dosage, and recording essential metrics like ingestion time and intervals. The post-session analysis provides users with comprehensive insights, fostering an evolving understanding of their cannabis experiences across diverse ingestion methods. This unified approach ensures consistency in data collection and analysis, promoting a holistic and personalized user experience irrespective of the chosen cannabis consumption method.

### Mechanical Function

### Setup

After the setup and dosage selection in the paired application, the chosen fresh bowl cartridge is identified by barcode or RFID and weighed by the scale, and that data transmits to the electronic device for later use. This cartridge is placed on the alignment shaft of the vape for use.

### Tip or Water Filter Accessory

The user can latch either the mouthpiece or the water filter accessory, both of which latch to the rest of the unit magnetically and continue the air path from the ovens to the user's mouth.

For the water filter accessory, the user may choose to add the latching magnetic stability base to the bottom of the unit to provide stability in the upright position when set down. The user fills the water pipe accessory to the appropriate level with either water or chipped ice for additional diffused vaporate cooling.

### Smart Base Accessory

The user may choose to add the smart base to the bottom of the unit to provide stability and to provide stand-alone controls and options through interaction with a rotary button/display that will provide a simplified subset of controls and information compared to the application on the electronic device. This smart base has its own reachable battery to avoid using any power from the vaporizer's battery.

### Use

The user begins the session by pressing the button on the side of the vaporizer unit. When the button is pressed and the user breathes in through the device, the vaporizer electrifies one oven coil while leaving the remaining oven coils off. Air rushes into the device to fill the partial vacuum created by the user. Air enters the device through a small, ring-shaped groove, around the flutes in the crash gasket. This air continues downward, flowing in the air gap between the oven unit and the body. This charge of air cannot travel further down, nor can air come from below because the oven unit is sealed to the wall with an O-ring/gasket at the insulating disk. The air charge must turn 90 degrees and enter the area around the posts and insulating disk. The air must turn 90 degrees again and process upward through the bores in the venturi disk above. The air charge enters the bore hole and encounters the oven coils. At this restriction point, the air speed increases as the air pressure drops, expanding the flow boundary layer at the edges of the bore hole, thereby reducing heat transfer to the surrounds of the oven coils. The single, active oven coil heats up to a predetermined, optimized temperature as the air charge passes through all the bores simultaneously. The air continues up through the cartridge screen and into the cartridge bores containing vaporate material. The bore with the active oven heats and vaporizes the cartridge bore above/aligned to it. That hotter stream of air containing the desired vapor, recombines with the other cold streams of air coming through the other, inactive ovens. The diffused air continues up to the user's mouth through either the cone tip or the water filter accessory. For the next dosage, another oven fires, and so on, in a star pattern to minimize unwanted heat flux between bores.

Note that each dosage results from the entire contents of one bore hole being heated in its entirety. This provides a repeatable, consistent dosage amount for each draw and therefore each session, in contrast to other vaporizers, where the vaporate material is contained in one, single large bore with one large oven, which as a mechanism cannot provide accurate, measured doses or sessions, because the entire mass is heated several times in a row, causing the vaporate produced to have an unpredictable logarithmic decay in concentration.

### Weigh Out, Report Effects

At the end of the session, the spent bowl cartridge is placed on the scale again to be re-identified and weighed out by the smart scale. That data transmits to the smart device and the program calculates the difference in mass between the fresh cartridge at weigh in and the spent cartridge at weigh out, thus providing the quantity of dosage that was vaporized and entered the lungs of the user.

Also, at this time and/or another time soon after the dosage when the dosage has taken full effect, the program will prompt the user to provide Qualitative feedback regarding the dose.

### Make Recoomendations

After accumulating sufficient data about the quantity and effects quality of several doses, the program on the electronic device will help the user optimize their desired experience through suggestion of dosage amount/rate, time of day/night for the doses, and highlighting of other strains/products to vaporize that may have desirable effects on the user.

What is claimed is:

1. A system for the dispersal of heated vaporates using a portable vaporizer containing a microprocessor running a subprogram to interact with an electronic device running a control program and a recharging unit comprising:
a. the electronic device comprising:
i. the control program to transmit user defined parameters to the microprocessor subprogram within the vaporizer and receive feedback on the execution of parameters from the subprogram;
ii. a running database for logging and comparing preprogrammed parameters;
b. the vaporizer comprising:
i. a mouthpiece unit;
ii. a removable vaporate cartridge unit containing at least one chamber configured to hold vaporate;
iii. a heating unit containing at least one oven, each of the at least one oven being controlled by the subprogram;
iv. an electronics unit comprising:
1. a rechargeable battery;
2. a printed circuit board; and
3. the microprocessor running the subprogram to control the heating unit; and send and receive communication with the control program within the electronic device;
v. a printed circuit board to connect the electronic elements;
c. the recharging unit having a base comprising:
i. two way communication means in communication with the control program;
ii. a recharging station comprising vaporizer receiving port;
iii. a vaporate cartridge station;
iv. a vaporate scale in communication with the vaporate cartridge station;
v. a rechargeable battery providing power to the vaporizer receiving port and the vaporate scale;
wherein the heating unit and electronics unit are removably contained within a body and the cartridge unit and the mouthpiece unit are removably affixed to the proximal end of the body;
wherein the control program is user programmed with vaporate specifics and the specifics are transmitted to the subprogram in the vaporizer for execution, the results of the execution being transmitted by to the control program to update the database.

2. The system of claim 1 wherein the vaporate specifics include at least one from the group comprising vaporate type; vaporate location; length of vaporate burn; temperature of vaporate burn, vaporate burn start time, vaporate burn end time and the vaporate specifics update the running database.

3. The system of claim 1 wherein each of the at least one vaporate cartridge chamber aligns with the at least one oven, thereby burning the vaporate associated with the at least one oven.

4. The system of claim 1 wherein the database further includes background information on each vaporate.

5. The system of claim 1 wherein a user weighs the vaporate cartridge before use and after use, the quantity of vaporate burned being transmitted to the control program for database update.

6. The system of claim 1 wherein the each of the at least one chamber in said vaporate cartridge aligns with each of the at least one oven in the heating unit to permit vapor to enter the mouthpiece unit during inhalation.

7. The system of claim 1 wherein the cartridge unit and the heating unit are maintained in alignment by an alignment shaft having V-shaped key that interacts with a central bore having V-shaped groove in the cartridge unit and the heating unit.

8. The system of claim 7 wherein the alignment shaft has at least one length section with a reduced diameter, the reduced diameter dimensioned to receive with heating elements.

9. The system of claim 1 wherein electronics unit and said heading unit are removably secured within the body of said vaporizer.

10. A system for the dispersal of heated vaporates using a portable vaporizer containing a microprocessor running a subprogram to interact with an electronic device running a control program and a recharging unit comprising:
a. the electronic device comprising:
i. the control program to transmit user defined parameters to the microprocessor subprogram within the vaporizer and receive feedback on the execution of parameters from the subprogram;
ii. a running database for logging and comparing preprogrammed parameters, the running database including background information on each vaporate;
b. the vaporizer comprising:
i. a mouthpiece unit;
ii. a removable vaporate cartridge unit having a center bore and containing at least one vaporate chamber configured to hold vaporate;
iii. a heating unit having a center bore and containing at least one oven, each of the at least one oven being controlled by the subprogram and each of the at least one oven aligning with each of the at least one vaporate chamber to burn only the vaporate associated with the aligning vaporate chamber and oven;
iv. an electronics unit comprising:
1. a rechargeable battery;
2. a printed circuit board; and
3. the microprocessor running the subprogram to control the heating unit; and send and receive communication with the control program within the electronic device;
v. a printed circuit board to connect the electronic elements;
vi. an alignment shaft having at least two diameters along its length and a V-shaped key on each diameter to maintain the heating unit and cartridge unit in alignment through interaction with a complementary V-shaped groove within a heating unit and cartridge unit bore;
c. the recharging unit having a base comprising:
i. two way communication means in communication with the control program;
ii. a recharging station comprising vaporizer receiving port;
iii. a vaporate cartridge station;
iv. a vaporate scale in communication with the vaporate cartridge station to enable a user to weigh the vaporate containing cartridge before and after use, the weight being transmitted to the control program;
v. a rechargeable battery providing power to the vaporizer receiving port and the vaporate scale;
wherein the heating unit and electronics unit are removably contained within a body and the cartridge unit and the mouthpiece unit are removably affixed to the proximal end of the body;
wherein the control program is user programmed with vaporate specifics and the specifics are transmitted to the subprogram in the vaporizer for execution, the results of the execution being transmitted by to the control program to update the database.

11. The system of claim 10 wherein the vaporate specifics include at least one from the group comprising vaporate type; vaporate location; length of vaporate burn; temperature of vaporate burn, vaporate burn start time, vaporate burn end time and the vaporate specifics update the running database.

12. The system of claim 10 wherein electronics unit and said heading unit are removably secured within the body of said vaporizer.
